(19) United States
(12) Patent Application Publication
(10) Pub. No.: US 2023/0416282 A1
(43) Pub. Date: Dec. 28, 2023

(54) ION-BASED METHODS FOR STABILIZING AND IMPROVING POROUS METAL-ORGANIC FRAMEWORK (MOF) MATERIALS FOR WATER HARVESTING FROM AIR AND RELATED APPLICATIONS

(71) Applicant: Northwestern University, Evanston, IL (US)

(72) Inventors: Joseph T. Hupp, Northfield, IL (US); Jian Liu, Wheeling, IL (US); Zhiyong Lu, Evanston, IL (US)

(21) Appl. No.: 18/035,876

(22) PCT Filed: Nov. 9, 2021
(86) PCT No.: PCT/US21/58554

§ 371 Date: May 8, 2023

Related U.S. Application Data
(60) Provisional application No. 63/111,451, filed on Nov. 9, 2020.

(51) Int. Cl. C07F 7/00; B01J 20/22

(52) U.S. Cl. CPC C07F 7/00; B01J 20/226

(57) ABSTRACT

Metal Organic Frameworks (MOFs), methods of using the MOFs for harvesting water from air, and methods of making the MOFs are provided. The water-harvesting MOFs include a metal organic framework molecule having positively charged, linker unsaturated nodes and a net positive electrical charge and charge-compensating anions contained within pores of the metal organic framework molecules. The charge-compensating ions are not anchored to the metal organic framework molecules via coordination bonds and, therefore, are able to move through the porous MOF structure when they are solvated in liquid water contained within the pores.

### CROSS-REFERENCE TO RELATED APPLICATIONS

The present application claims priority to U.S. provisional patent application No. 63/111,451 that was filed Nov. 9, 2020, the entire contents of which are incorporated herein by reference.

### REFERENCE TO GOVERNMENT RIGHTS

This invention was made with government support under DE-FG02-08ER15967 awarded by the Department of Energy. The government has certain rights in the invention.

### BACKGROUND

Porous materials, such as metal-organic frameworks (MOFs), have shown promising water-vapor adsorption properties that are relevant for many applications, including heat pumps, chillers, and water harvesting from air, depending on the relative humidity (RH) where the water uptake occurs. Chemical and mechanical hydrolytic stability of MOFs in the presence of water and during water removal are prerequisites for using MOFs in these kinds of devices. For water-harvesting from air, humidity is spontaneously captured from air and condensed as liquid water within the network of nanostructured pores defining the interior space of a crystalline MOF. A change in external relative humidity, typically engendered by a modest change in temperature (for example, for night versus day), ideally causes the MOF to release the captured water, setting the stage for additional vapor-capture/water-release cycles. An ideal material for harvesting water from air of finite humidity will exhibit: 1) a high water-uptake capacity, which is typically achieved by relying on comparatively large pores (e.g., a few nanometers in diameter); 2) chemical hydrolytic stability during water-vapor uptake and mechanical stability (for example, against pore-collapsing capillary forces) during water release, which is typically achieved by comparatively small pores, together with water-substitution-resistant bonds between MOF nodes (metal-ion-containing, inorganic nodes) and their organic linkers; and 3) depending on the application, complete or nearly complete uptake of water vapor and reversible storage as liquid water, which is typically achieved by relying on comparatively small pores (e.g., diameters of ˜1 nm or less). Thus, inherent contradictions in MOF architecture optimization exist for these three sets of behavior; large pores are desirable for meeting the first requirement, while small pores are desirable for satisfying the second and third requirements.

### SUMMARY

MOFs, methods of using the MOFs for harvesting water from air, and methods of making the MOFs are provided.

One example of a metal organic framework includes: a metal organic framework molecule having positively charged, linker unsaturated nodes and a net positive charge; water contained within pores of the metal organic framework molecule; and charge compensating anions that are not bonded to the linker unsaturated nodes via coordination bonds and that have freedom of motion through the pores of the metal organic framework.

One example of a method for harvesting water from an atmosphere containing vapor-phase water molecules uses metal organic frameworks that include: metal organic framework molecules having positively charged, linker unsaturated nodes and a net positive charge; and charge compensating anions that are not bonded to the linker unsaturated nodes via coordination bonds and that have freedom of motion through the pores of the metal organic framework. The method includes the steps of: exposing the metal organic frameworks to an atmosphere at a temperature at which vapor-phase water molecules condense as liquid water within the pores of the metal organic framework molecules, wherein the water-soluble ions are solubilized by the liquid water; increasing the temperature of the metal organic frameworks to a temperature at which the water molecules are released from the pores of the metal organic framework molecules, decreasing the relative humidity of the atmosphere to a relative humidity at which the water molecules are released from the pores of the metal organic framework molecules, or both; and collecting the released water molecules.

One example of a method of making charge-compensated metal organic frameworks includes the steps of: synthesizing metal organic framework molecules having linker unsaturated nodes and an initial set of ligands on the linker unsaturated nodes; dispersing the metal organic framework molecules in a solution comprising a salt that dissociates into cations and charge compensating anions, wherein the cations form coordination bonds to the linker unsaturated nodes to impart a net positive charge to the metal organic framework molecules, and the charge-compensating anions remain solvated; and separating the positively charged metal organic frameworks and the charge-compensating anions from the solution; and drying the metal organic framework molecules to provide charge-compensated metal organic frameworks comprising positively charged metal organic framework molecules and charge compensating anions dispersed within pores of the positively charged metal organic framework molecules.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIGS. 1A-1B show N₂ adsorption isotherms (FIG. 1A) and pore-size distributions (FIG. 1B) of NU-1000-F and NU-1000-FF-Cl samples treated with various non-formate-generating solvents, respectively. FIGS. 1C-1D show N₂ adsorption isotherms and pore-size distributions of NU-1000-F and NU-1000-FF-Cl samples treated with various non-formate-generating solvents, respectively.

FIGS. 2A-2E show a crystal structure comparison between NU-1000-F and NU-1000-FF-Cl. FIGS. 2A and 2C show the micropores and mesopores viewed along the c axis: mesopore size was measured by the distance between Zr atoms on each opposite sites, the edge of triangle pore is the length between the nearest Zr atoms on the edge, and the vertical length is the distance between Zr atom at the vertex and the opposite pyrene plane; FIG. 2B shows the Zr₆ node of NU-1000-F; FIGS. 2D and 2E show the Zr₆ node of NU-1000-FF-Cl viewed from different angles, indicating the specific locations of water molecules around the node.

FIG. 3 shows an illustration of NU-1000-FF-Cl (2) synthesis from as-synthesized sample (1). For simplicity, formates and benzoates are represented as bidentate ligands, but might be monodentate in the case of benzoate.

FIG. 4 shows N₂ adsorption isotherms for MOF-808-F, MOF-808-Cl, and MOF-808-Br.

FIG. 5 shows water sorption isotherms comparing as-synthesized MOF-808-F to MOF-808-Cl and to a MOF-808 from which both the Cl and formate ligands have been removed (MOF-808-FF).

FIG. 6 shows water sorption isotherms for MOF-808-Cl through 5 cycles.

FIG. 7 shows water sorption isotherms comparing as-synthesized NU-1000-F to NU-1000-Cl and to NU-1000 from which both the Cl and formate ligands have been removed (NU-1000-FF).

FIG. 8 shows water sorption isotherms for NU-1000-Cl through 5 cycles.

FIG. 9 shows the siting of charge-compensating halide ions in MOF-808 after each of several nonstructural anionic formate ligands has been replaced by a pair of charge-neutral aqua ligands. Replacement renders the overall framework cationic.

FIGS. 10A and 10B show isotherms for water uptake and release by: MOF-808-F (FIG. 10A), 3 cycles; MOF-808-Br (FIG. 10B), cycles 1-5 and 21.

### DETAILED DESCRIPTION

MOFs, methods of using the MOFs for harvesting water from air, and methods of making the MOFs are provided.

MOFs are hybrid, crystalline, porous materials made from metal-ligand networks that include inorganic nodes chemically coordinated by multitopic organic linkers. The inorganic nodes, which are vertices in the framework, can be composed of metal ions or clusters. By convention, carboxylates (or other linker terminal groups or atoms) are often represented as components of the nodes. The nodes are connected by coordination bonds to the organic linkers, which commonly contain carboxylate, phosphonate, pyridyl, imidazolate and/or other azolate functional groups. The coordination sites on the nodes may be saturated with organic linker connections. However, MOFs may also have nodes with coordination sites that are not coordinated to organic linkers. Such nodes, which are referred to herein as linker unsaturated nodes, may be coordinated to ligands at the unsaturated sites. These ligands may bind to a single node atom (e.g., metal atom) or may bridge metal atoms within a node. However, unlike the organic linkers, the ligands do not bridge nodes with in the MOF. MOFs are commonly referenced based on the metal in their nodes. For example, a “zirconium MOF” is a MOF with zirconium atoms or clusters in its nodes.

The water-harvesting MOFs include a metal organic framework molecule having positively charged, linker unsaturated nodes and a net positive electrical charge; and water-soluble charge-compensating anions contained within pores of the metal organic framework molecules. The charge-compensating anions are not ligated and, therefore, are able to move through the porous MOF structure when they are solvated in liquid water contained within the pores.

The MOFs have high water-uptake capacities and are chemically hydrolytically stable during water-vapor uptake and mechanically stable during water release. While the inventors do not intend to bound to any particular theory of the invention, the observed stability during water uptake and release can be attributed to the presence of the positive charges anchored at the nodes of the MOF's molecular framework and the water-solvated charge-balancing ions that retain freedom of motion through the porous framework because they are not chemically grafted or otherwise strongly bound to the charged framework. The freely moving, solvated charge-compensating ions within the MOF may serve to mitigate the, otherwise substantial, mechanical strain imposed upon the charged metal organic framework molecule as water is released, enabling the MOF to resist capillary-force-driven channel-collapse and preserving its initial water uptake capacity over the course of multiple uptake and release cycles. Moreover, the ability of the charge-compensating ions to be solvated by incoming water molecules may serve to facilitate water uptake even in lower relative-humidity environments, while the charges anchored to the framework molecule may similarly serve to facilitate water uptake and release at a lower relative-humidity than would be needed for a charge-neutral metal organic framework molecule.

The water-harvesting MOFs can be used to obtain potable liquid water by direct harvesting from low or moderate humidity air, using zero or minimal expenditure of non-ambient energy. For example, natural day to night humidity and/or temperature cycles for the atmosphere could serve as sources of ambient energy. The water-harvesting MOFs also can be used in porous-materials-based heat pumps, chillers, and water purifiers.

A net positive electrical charge may be imparted to a metal organic framework molecule that lacks a net positive electrical charge by reacting pre-existing ligands on the nodes with a salt in an aqueous or non-aqueous solution, whereby the salt dissociates to form a cation that binds to the node and a charge compensating anion that remains unligated. Suitable salts include acids, including inorganic acids, that dissociate into a proton that protonates one or more nodes and an anion. Suitable strong acids include those the provide a halide ion as a charge-compensating anion. These include hydrochloric acid (HCl), hydrobromic acid (HBr), and hydroiodic acid (HI).

The pre-existing ligands are the initial set of ligands coordinated to the nodes. These initial ligands, which may be neutral or negatively charged, may be present by virtue of the MOF synthesis process (for example, the ligands may be modulators used to control the rate of crystal growth) and/or by virtue of a process used to activate the MOF and/or to remove modulator ligands. For example, hydroxo, aqua, oxo, benzoate, formate, acetate, trifluoroacetate ligands, and combinations of two or more thereof are commonly present on the nodes of as-synthesized and/or activated MOFs.

The reaction with the salt is desirably carried out using a solvent that does not generate ligands that compete with the salt cations for coordination at the node. By way of illustration only, dimethyl sulfoxide (DMSO), N-methyl-2-pyrrolidone (NMP), and N,N-dimethylacetamide (DMA) may be used. A solvent-exchange can be carried out in order to replace the synthesis solvent with the new reaction solvent, prior to the salt (e.g., acid) treatment. The as-synthesized, or as-activated MOFs may be exposed to the reaction solution for a time sufficient to impart a net positive electrical charge to the metal organic framework molecule. Optionally, the reaction solution may be heated to facilitate the reaction. The MOFs and their associated charge-compensating anions, which are now present in the pores of the metal organic framework molecules, are then removed from the reaction solution and dried.

It is not necessary for all of the pre-existing ligands to be converted into electrically charged ligands, but it is desirable for the majority (i.e., at least 50 mol. %) of the ligands on the nodes to be converted (for example, protonated). Thus, in various embodiments of the water-harvesting MOFs, positively charged ligands account for at least mol. %, at least 60 mol. %, at least 70 mol. %, at least 80 mol. %, at least 90 mol. %, at least mol. %, at least 99 mol. %, or 100% of the ligands at the linker unsaturated nodes. However, it is also possible for the positively charged ligands to make up less than 50 mol. % of the node ligands.

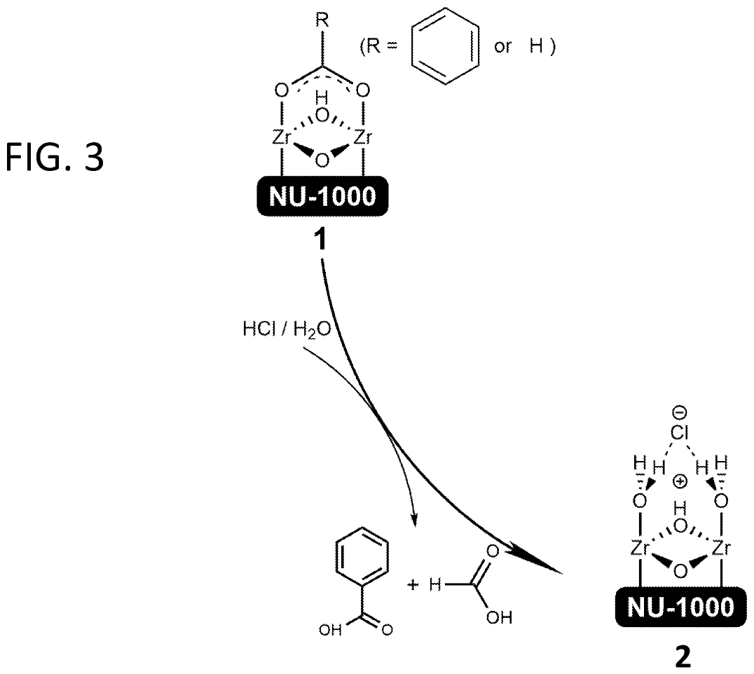

For purposes of illustration, FIG. 3 shows the reaction scheme for converting pre-existing benzoate or formate ligands on zirconium atoms of a zirconium MOF (NU-1000) into positively charged (e.g., protonated) ligands using HCl as a reactant to produce a MOF having protonated nodes and a net positive electrical charge that is balanced by mobile chloride ions. More details regarding this reaction scheme are provided in the Example.

The water-harvesting MOFs may be formed from a wide variety of MOFs having linker unsaturated nodes that can be protonated or otherwise imparted with a positive charge. Such MOFs include, but are not limited to, zirconium MOFs, hafnium MOFs, cerium MOFs, chromium MOFs, aluminum MOFs, indium MOFs, and titanium MOFs. Specific examples of MOFs that can be used to make the water-harvesting MOFs include zirconium MOFs, such as 8-connected NU-1000, 6-connected MOF-808, NU-901, UiO-6X series MOFs, NU-1008, and NDC-NU-1000. The hafnium and cerium analogs of these and other zirconium MOFs can also be used. NU-1000 is a hierarchically porous MOF featuring 1D channels, employing 1,3,5,8-(p-benzoate)pyrene (TBAPy₄⁻) units as linkers, and offering an 8-connected node having the composition Zr₆(μ₃-O)₄(μ₃-OH)₄(H₂O)₄(OH)₄⁸⁺ (with eight linker-based carboxylate anions balancing the 8⁺ charge of the node and leaving the overall framework charge-neutral). This MOF and the others are well-known in the art. More detailed information about the structures of these MOFs can be found, for example, in PCT publications WO 2020/086496 and WO 2017/213871 and in the following references: Furukawa et al., Water adsorption in porous metal-organic frameworks and related materials. J Am Chem Soc 2014, 136 (11), 4369-81; Peters et al., Site-Directed Synthesis of Cobalt Oxide Clusters in a Metal-Organic Framework. ACS Appl Mater Interfaces 2018, 10 (17), 15073-15078; Mondloch et al., Vapor-phase metalation by atomic layer deposition in a metal-organic framework. J Am Chem Soc 2013, 135 (28), 10294-7; and Garibay et al., Synthesis and functionalization of phase-pure NU-901 for enhanced CO₂ adsorption: the influence of a zirconium salt and modulator on the topology and phase purity. CrystEngComm 2018, 20 (44), 7066-7070.

When the water-harvesting MOFs are exposed to a water vapor-containing atmosphere at a suitable temperature, water molecules diffuse into the pores of the metal organic framework molecules where they condense into liquid water. The charge-compensating anions, which—unlike the positively charged ligands—are not bound to the MOF via coordinate covalent bonds, become solvated in the liquid water and are able to move through the liquid-filled porous MOF structure. The optimal temperature for water uptake will depend on the particular MOF being used. However, typically temperatures in the range from about 5° C. to about 25° C. are sufficient to achieve water uptake and condensation.

The liquid water can then be released from the water-harvesting MOFs through evaporation by heating the MOFs to a temperature and/or reducing the relative humidity (RH) of the external environment. The optimal temperature or RH for water release will depend on the particular MOF being used. However, typically temperatures in the range from about 30° C. to about 60° C. are sufficient to achieve water release. The release of water molecules for the MOFs can be achieved, for example, by changing the external relative humidity of the surrounding environment. Such a change may be engendered by a modest increase in temperature. For example, a typical temperature accompanying the change from night to day may cause the MOF to release the captured water, setting the stage for additional vapor-capture/water-release cycles. By way of illustration the MOFs can be used to take up water at temperatures in the range from 5° C. to 25° C. and RH conditions in the range from 20% to 35% and release water at temperatures in the range from 30° C. to 50° C. and RH conditions in the range from 5% to 20%. Thus, the MOFs are useful for water harvesting even in deserts climates.

The released water can then be collected and the water-harvesting MOFs can then be reused for additional water-harvesting cycles (e.g., two or more cycles, five or more cycles, 10 or more cycles, or more). Notably, the water-harvesting MOFs exhibit useful water uptake levels even in atmospheres with low to moderate RH. For example, the water-harvesting MOFs can be used in an atmosphere with an RH in the range from 10% to 60%. Further, uptake and release can be reversibly accomplished over narrow ranges of change in relative humidity, with pertinent relative RHs centering on low (15 to 20%) or moderate (50 to 60%) RH values. Thus, the water-harvesting MOFs described herein are better suited than the parent MOFs from which they are made for applications in harvesting water from air with minimal non-ambient energy consumption.

### Example

This example illustrates water uptake from a low humidity environment using the well-known zirconium-node-based MOFs NU-1000 and MOF-808. As initially synthesized, both MOFs presented, on their zirconium nodes, non-structural aqua and hydroxo ligands coordinated to zirconium atoms, with the nodes also chemically coordinating to the multitopic organic linkers needed to define the high-porosity frameworks. It should be noted, however, that the NU-1000 and MOF-808 MOFs are used as illustrative examples only and that the procedures described in this Example can also be used on other MOFs having non-structural aqua and hydroxo ligands, or even other ligands, on their metal nodes.

As discussed in greater detail in the Experimental section, the MOFs were treated with aqueous HCl and aqueous HBr, well-known strong acid (i.e., a fully dissociating acids that exists in water as separate cations (solvated protons (H+)) and anions (solvated chloride ions (Cl−) or bromide ions (Br−)). Atomically precise, single-crystal X-ray structural measurements and other measurements established that the protons were binding to the non-structural hydroxo or other ligands and imparted an overall positive charge to the framework. In contrast, in the presence of the pore-filling solvent, the charge-compensating chloride ions resided in the pores in solvated form, and did not form coordinated covalent bonds to the framework. As such, the chloride ions were capable of: a) moving through the water-containing pores; and b) changing their locations in response to water removal from, or condensation in, the pores of the metal organic framework molecules. This freedom of movement, together with hydration of the charge-balancing ions, appeared to mitigate the otherwise substantial mechanical strain upon the charged framework as water was extracted. Presumably, the mitigation of strain was what enabled the framework to resist capillary-force-driven channel-collapse—in turn, preserving initial water uptake capacity over the course of additional uptake and release cycles. The ability of the charge-compensating ions to interact with (i.e., be solvated by) incoming water molecules may account for the sharp water uptake at lower relative-humidity. The charges (protons in this illustrative example) attached to the framework may similarly serve to favor water uptake and release at lower relative-humidity than required by the charge-neutral, parent framework.

In contrast to as-synthesized (“parent”) MOF-808 and NU-1000, the electrically charged metal organic framework molecules with their charge-balancing chloride anions (referred to herein as MOF-808-Cl, MOF-808-Br, and NU-1000-Cl) showed reversible or nearly reversible water vapor uptake and release through five cycles of systematic increase and decrease of relative humidity. For MOF-808-Cl, at ambient temperature, reversible water uptake occurred mainly between RH 15 and 20%, with uptake capacity reaching 0.53 g g⁻¹ (grams of water per gram of sorbent). For NU-1000-Cl, nearly reversible water uptake occurred mainly between RH 50 and 60%, with a capacity maximum of 1.0 g

### Experimental

Synthesis of NU-1000 (powder/single crystal, denoted as as-syn-NU-1000). The synthesis of bulk microcrystalline powder and single-crystal as-syn-NU-1000 was performed according to a recently reported procedure. (Islamoglu, T. et al., CrystEngComm 2018, 20 (39), 5913-5918.) Briefly, 98 mg ZrOCl₂·8H₂O and 2 g benzoic acid were dissolved in N,N′-dimethylformamide (DMF) (8 mL) and then incubated in an oven at 100° C. for 1 h. After cooling down to room temperature, 40 mg H₄TBAPy and 40 uL trifluoroacetic acid (TFA) were added and sonicated for 10 min. The suspension was kept in an oven at 100° C. for 18 h, yielding yellow polycrystalline as-syn-NU-1000. For single-crystal synthesis, 70 mg ZrCl₄ and 2 g benzoic acid were dissolved in N,N′-diethylformamide (DEF) (6 mL) and then incubated in an oven at 100° C. for 1 h. In the meantime, 40 mg H₄TBAPy was added to 4 mL DEF and heated to 100° C. for 1 h. (Thus, the molar ratio of benzoic acid to linker was roughly 230:1.) After cooling to room temperature, H₄TBAPy solution and 40 uL TFA were added to the pre-made Zr-node-containing solution and sonicated for 10 min. The yellow suspension was kept in an oven at 120° C. for 24 h to afford ˜120 microns as-syn-NU-1000 single crystals.

Formate-containing NU-1000 (powder/single crystal, denoted as NU-1000-F). About 50 mg of as-syn-NU-1000 was washed sufficiently with DMF at least 5 times over the course of 24 hr to remove trapped modulators and linkers from the pores. Then the yellow solid was dispersed in a solution of 15 mL of DMF and 0.6 mL of 8M aqueous HCl. The mixture was heated in an oven at 100° C. for 18 h. After cooling to room temperature, the solid was isolated by centrifugation and washed with DMF over the course of 8 hr (4×15 mL). For porosity characterization, the powder sample was solvent-exchanged with acetone three times (15 mL each) (soaked ˜1 h between washes) and soaked in acetone for an additional 16 h. The NU-1000-F sample was collected by centrifugation and dried in a vacuum oven at 80° C. for 1 h, and thermally activated at 120° C. for 18 h under dynamic vacuum to yield pale yellow powder.

Formate-free (“FF”) NU-1000 containing chloride (powder; denoted as NU-1000-FF-Cl). About 50 mg of as-syn-NU-1000 was solvent-exchanged with DMSO at least times over the course of 24 hr. The yellow precipitate was then dispersed in 15 mL of DMSO and 0.6 mL of 8M aqueous HCl. The mixture was kept at room temperature for 18 h. The yellow polycrystalline material was collected into centrifuge tubes and washed with DMSO over the course of 8 hr (4×15 mL). For porosity characterization, the sample was solvent-exchanged with acetone three times (15 mL each, with ˜1 h of soaking between washes) and soaked in acetone for an additional 16 h. The NU-1000-FF-Cl sample was collected by centrifugation, dried in a vacuum oven at 80° C. for 1 h, and then thermally activated at 120° C. for 18 h under dynamic vacuum to yield yellow powder.

Formate-free NU-1000 containing chloride (single-crystals). 40 mg crystals of NU-1000-F were solvent exchanged with DMSO over the course of 24 h (6×10 mL) and kept in 12 mL DMSO. Aqueous HCl (0.5 mL, 8M) was added into the solution. The mixture was swirled occasionally and kept at room temperature for 18 h. The NU-1000-FF-Cl crystals were then washed with DMSO over the course of 8 h (5×10 mL) and kept in DMSO and not thermally evacuated before single-crystal X-ray diffraction data were collected from a hexagonal-rod-shaped crystal of 0.1 mm length and 0.03 mm width.

Formate-Free MOF-808 Containing Chloride or Bromide (Denoted as MOF-808-FF-CL and MOF-808-BR).

The synthesis of bulk microcrystalline powder MOF-808-F was performed according to a reported procedure (J Am Chem Soc 2014, 136 (11), 4369.). Briefly, 0.48 g ZrOCl₂·8H₂O and 0.33 g 1,3,5-1,3,5-tricarboxybenzene were dissolved in DMF (60 mL), and then 60 mL formic acid was added. The solution was kept in an oven at 100° C. for 48 h, yielding white polycrystalline. The white sample was washed with DMF for at least 5 times to remove trapped modulators and linkers from the pore (50 mL each; soaking ˜1 h between washes), and then solvent-exchanged with acetone three times (50 mL each; soaking ˜1 h between washes) and soaked in acetone for an additional 16 h. MOF-808-Formate sample was collected by centrifugation, dried in a vac. oven (80° C. for 1 h), and activated at 120° C. for 18 h under dynamic vacuum to yield white powder.

About 50 mg of as-synthesized MOF-808-F was solvent-exchanged with DMSO at least 5 times over the course of 24 hr. Then, the MOF was dispersed in 15 mL of DMSO and 0.6 mL of 8M aqueous HCl or HBr. The mixture was kept at room temperature overnight. The material was collected into centrifuge tubes and washed with DMSO over the course of 8 h (4×15 mL). For porosity characterization, the sample was solvent-exchanged with acetone three times (15 mL each) (soaked ˜1 h between washes) and soaked in acetone for an additional 16 h. The sample was collected by centrifugation and dried in a vacuum oven at 80° C. for 1 h, and then thermally activated at 120° C. for 18 h under dynamic vacuum to yield products. (Note, the process by which MOF-808 was synthesized used a formamide-based solvent. As a result, the as-synthesized MOFs had formate ligands at the nodes. For this reason, the as-synthesized MOF is denoted MOF-808-F.)

Synthesis of MOF-808-Br. About 40 mg of as-synthesized MOF-808-F sample was solvent-exchanged with dioxane for at least 5 times over the course of 24 h to remove DMF from the pores. The white precipitate was then dispersed in 12 mL of dioxane and 0.24 mL of 8M aqueous HBr. The mixture was kept at room temperature for 18 h. The white polycrystalline material was collected into centrifuge tubes and washed with dioxane over the course of 8 h (4×12 mL). The powder sample was further solvent-exchanged with acetone three times (15 mL each, with ˜1 h of soaking between washes) and soaked in acetone for an additional 16 h. The MOF-808-Br sample was collected by centrifugation and dried in a vacuum oven at 80° C. for 1 h, and then thermally activated at 120° C. for 18 h under dynamic vacuum to yield grey powder.

Synthesis of MOF-808-I. About 40 mg of as-synthesized MOF-808-F sample was solvent-exchanged with dioxane for at least 5 times over the course of 24 h to remove DMF from the pores. The white precipitate was then dispersed in 12 mL of dioxane and 0.24 mL of 8M aqueous HI. The mixture was kept at room temperature for 18 h. The white polycrystalline material was collected into centrifuge tubes and washed with dioxane over the course of 8 h (4×12 mL). Further washings were conducted when ¹H NMR spectra of digesting sample showed the incomplete removal of formate. The powder sample was further solvent-exchanged with acetone three times (15 mL each, with ˜1 h of soaking between washes) and soaked in acetone for an additional 16 h. The MOF-808-I sample was collected by centrifugation and dried in a vacuum oven at 80° C. for 1 h, and then thermally activated at 120° C. for 18 h under dynamic vacuum to yield brown powder.

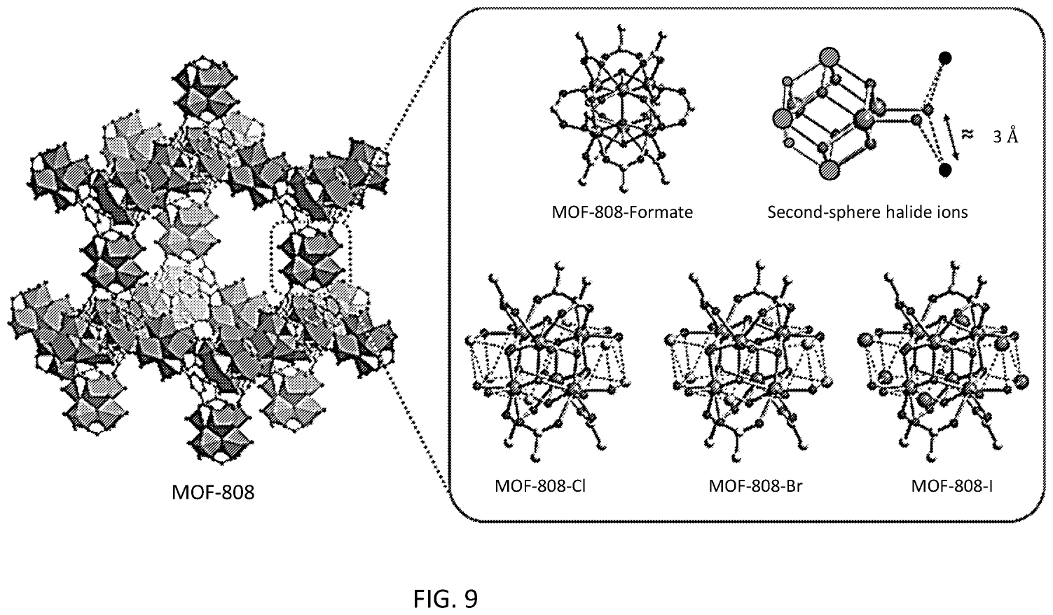

FIG. 9 shows the siting of charge-compensating halide ions in MOF-808 after each of several nonstructural anionic formate ligands has been replaced by a pair of charge-neutral aqua ligands. Replacement renders the overall framework cationic. (MOF-808-Cl—lower left; MOF-808-Br—lower middle; MOF-808-I—lower right.)

BET Surface Area Measurements. N₂ isotherms were measured with a Micromeritics Tristar II instrument. Measurements were performed at 77 K with the temperature held constant with a liquid nitrogen bath. Consistency criteria were adapted to choose an appropriate pressure range for BET surface-area calculations. (Gomez-Gualdron, D. A. et al., J Am Chem Soc 2016, 138 (1), 215-24.)

¹H NMR Spectroscopy. Samples were prepared by weighing 2 mg of MOF into a 1.5 mL vial. About 3 to 4 drops of 0.1 M NaOD in D₂O digestion medium was then added to the vials. The vials were capped and inverted 2 or 3 times before sonicating for 30 min. This procedure dissolved only the organic portion of the MOF (linker and modulator); the inorganic component precipitated as zirconium oxide or hydroxide. To the mixture, 17 drops of D₂O were added. Then the mixture was centrifuged and the clear supernatant solution was transferred to an NMR tube. ¹H NMR spectra were recorded with a Bruker Avance DPX-500 NMR spectrometer (500 MHz) and the number of scans was set to 64.

Diffuse reflectance infrared Fourier transform spectra (DRIFTS) were recorded on a Nicolet 7600 FT-IR spectrometer equipped with an MCT/A detector. Samples diluted in KBr were measured with a KBr background and kept at each temperature under Ar purge (samples prepared in atmosphere). The spectra were collected at 1 cm⁻¹ resolution and 32 scans were averaged over the spectral window of 675-4000 cm⁻¹. All samples were activated samples, evaluated after BET surface area characterization.

Water Sorption Isotherms. Water isotherms were measured on a micromeritics 3Flex, and the water uptake in cm 3 g⁻¹ units was plotted as a function of relative pressure (P/P₀). Prior to the water adsorption measurements, water (analyte) was flash frozen under liquid nitrogen and then evacuated under dynamic vacuum at least 3 times to remove any gases in the water reservoir. The P₀ was checked at room temperature before measurement. The measurement temperature was controlled with a micromeritics temperature controller. Activation of MOFs was performed under a dynamic vacuum for 12 h on SVP (SmartVapPrep from Micromertics) at 120° C. (2° C./min).

### Results

A synthesis protocol for MOF-activation to prevent further node-ligation of formate, to remove already-installed formate and initially present benzoate ions, and to impart a positive net charge to the frameworks and to introduce charge-compensating (also referred to as charge-balancing) chloride ions was developed. Two approaches were used: 1) replacing DMF with a non-formate-generating solvent in the putative chemical activation step at 100° C., and 2) retaining DMF in the second step (i.e., activation step), but lowering the activation temperature, with the goal of avoiding DMF decomposition and renewed formate formation. For the first approach, each of three non-formate-generating solvents was examined as an alternative to DMF: dimethyl sulfoxide (DMSO), N-methyl-2-pyrrolidone (NMP), and N,N-dimethylacetamide (DMA). Before acid treatment, the as-syn-NU-1000 sample was solvent-exchanged with corresponding solvent at least five times over the course of 24 h. From solution-phase ¹H NMR spectra obtained by digesting samples in NaOD/D₂O, the amount of benzoate present was lowered to a level that was undetectable, and the initially-installed formate was close to completely removed, i.e., 0.06 formate ligands remained per node following treatment with DMSO/HCl and 0.08 after treatment with NMP/HCl. Treatment with DMA/HCl was less effective, leaving 0.7 formates per node.

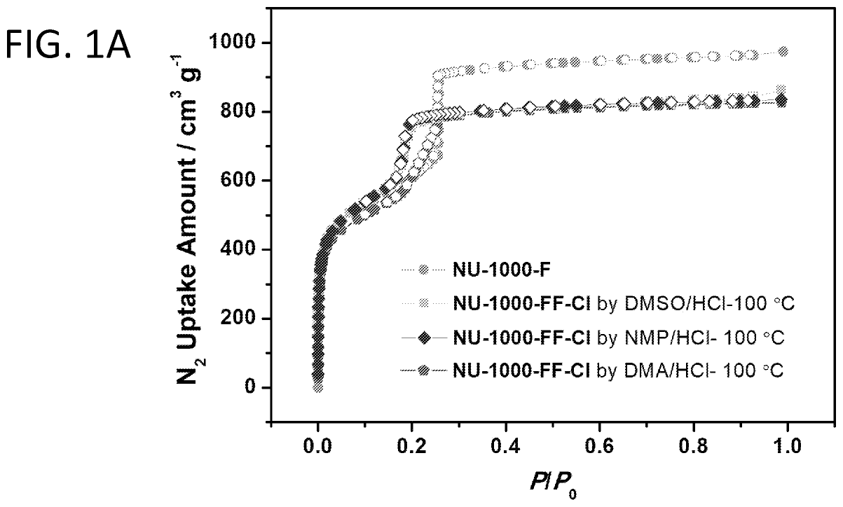

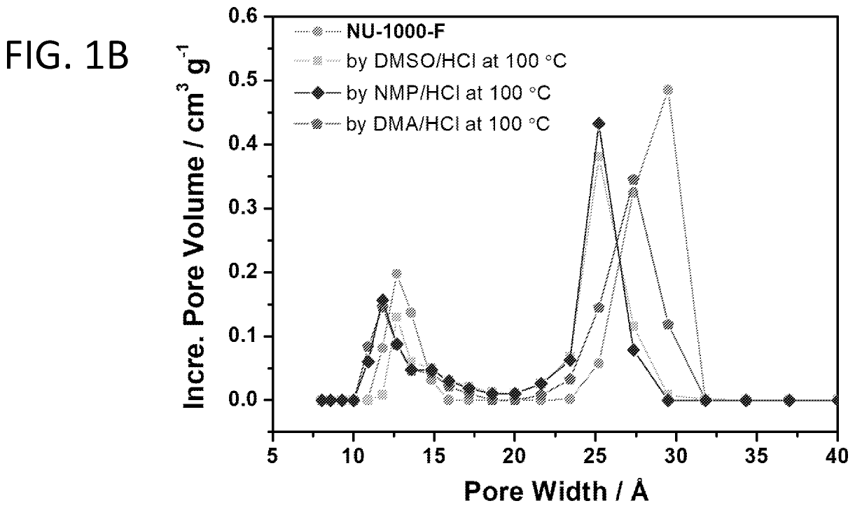

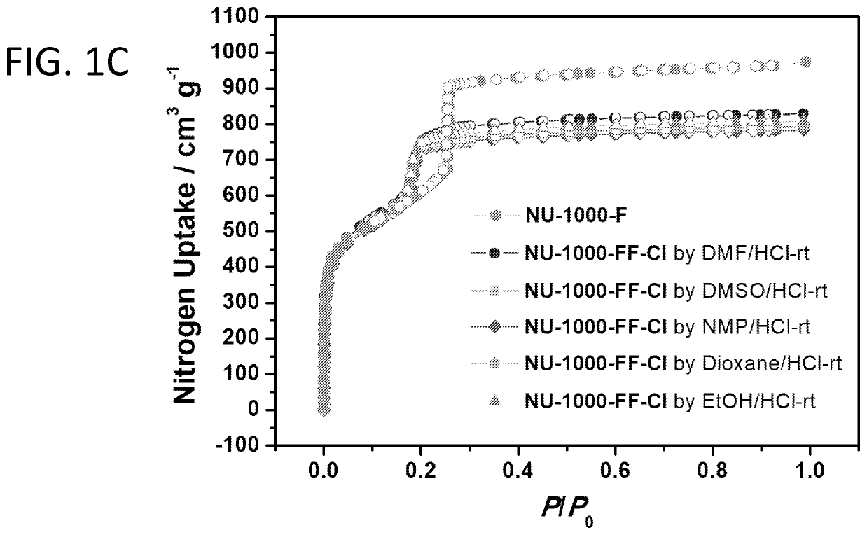

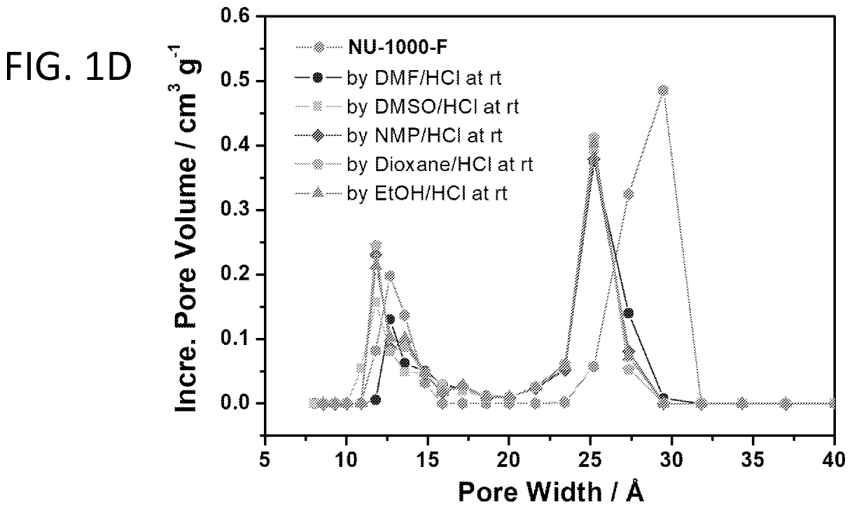

N₂ adsorption isotherms (FIG. 1A) for DMSO/HCl-treated and NMP/HCl-treated samples yielded BET surface areas of 2020 m² g⁻¹ and 2070 m² g⁻¹, respectively. These values are slightly lower than that of NU-1000-F (2150 m² g⁻¹), where all samples were thermally activated at 80° C. and then 120° C., as detailed in the Experimental Section. Isotherms for the DMSO/HCl-treated and NMP/HCl-treated samples were closely similar to each other, but differed somewhat from that for NU-1000-F. The former displayed slightly lower plateau values, implying slightly smaller total pore volumes. They also displayed slightly lower P/P₀ values for the mesopore step for NU-1000, i.e. a drop from ˜0.25 to ˜0.20. The latter translated to a contraction of the N₂-accessible width of the hexagonal-channel-shaped pore to 2.5 nm (from 3.1 nm); see FIG. 1B, which indicates a proportional contraction in the width of the triangular micropore. The contraction behavior was unanticipated, especially in view of the removal of chelated formate from the nodes during HCl treatment.

The second approach, i.e., treatment with DMF+aq. HCl at room temperature, yielded similar changes in pore diameters and total pore volume, although with slightly less effective elimination of formate, as 0.2 formates per node were retained. As illustrated in FIGS. 1C-1D, several other solvents, in combination with aq. HCl at room temperature, were effective in removing formate, protonating the framework, and introducing charge-compensating chloride ions. Formate anion removal was matched by incorporation of chloride. These samples are collectively referred to, therefore, as NU-1000-FF-Cl samples.

Further investigation, as detailed below, revealed that the decrease of porosity in NU-1000-FF-Cl was not indicative of structure collapse, but instead was a consequence of reversible pore contraction, formate removal, charge-balancing incorporation of chloride in place of formate, and other alterations. Given the effectiveness of DMSO for formate removal, DMSO/HCl-rt treated NU-1000-FF-Cl was chosen as the illustrative sample for the water harvesting experiments.

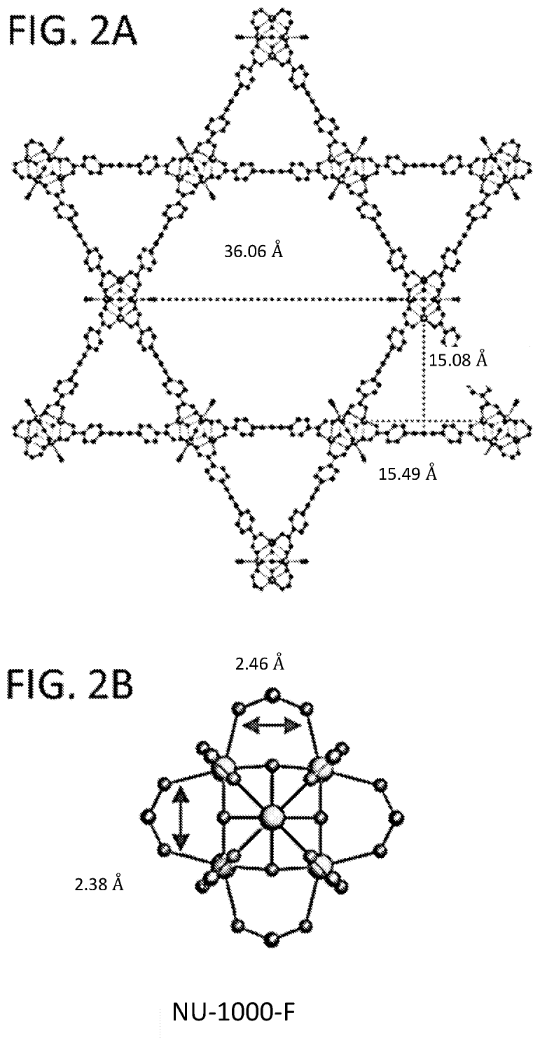

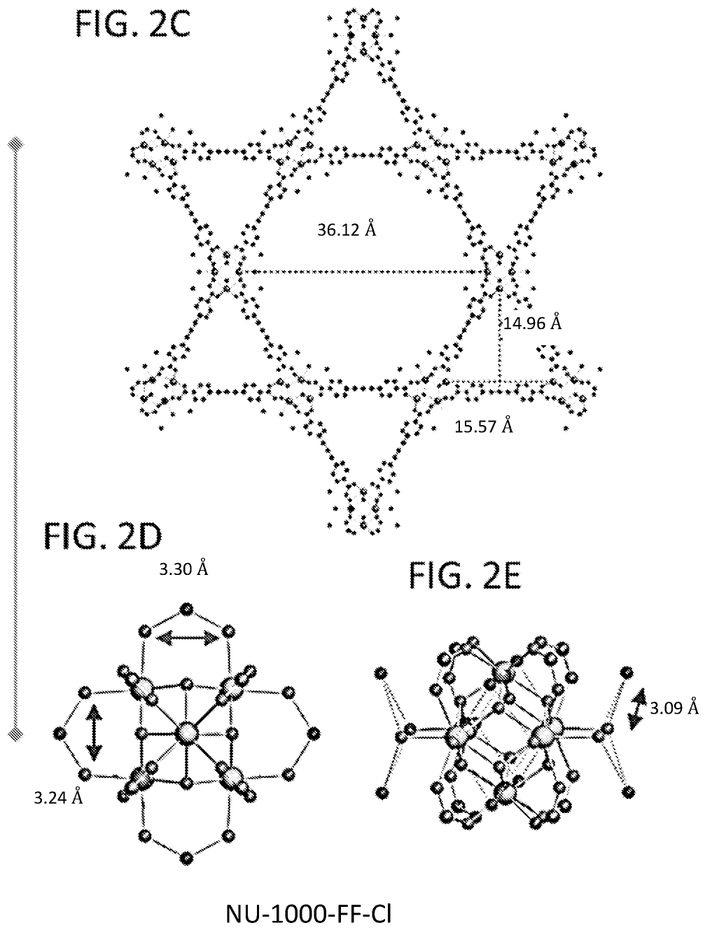

Single-crystal structures. The structure of a sample of NU-1000-FF-Cl obtained by treating as-syn-NU-1000 with DMSO+aq. HCl at room temperature, followed by soaking in DMSO was determined via single-crystal X-ray diffraction, (Thus, the void spaces within the MOF contained free DMSO.) The single-crystal structure of NU-1000-F was also re-measured. As shown in FIGS. 2A-2B, the measurement for NU-1000-F revealed the presence of node-coordinated formate ions, with each formate linking a pair of zirconium ions. Consistent with separate measurements (above) indicating loading of ˜3 formates out of an anticipated maximum of 4, the X-ray-determined occupancy of the carbonyl carbon of formate was less than 1. It was concluded that, on average, the nodes of eight-connected NU-1000-F were nonstructurally ligated by ˜3 formates and one hydrogen-bond-linked OH/OH₂ pair. The unit cell parameters of NU-1000-FF-Cl were: a=b=39.6588 (6) Å, c=16.3927 (3) Å, α=β=90°, γ=120°, V=22329 (2) Å³, which were similar to those of NU-1000-F (a=b=39.568(4) Å, c=16.5440(17) Å, α=β=90°, γ=120°, V=22432(5) Å³).

Comparison of the solvent-filled structures of NU-1000-FF-Cl and NU-1000-F yielded essentially identical pore diameters (see FIGS. 2A and 2C) and essentially identical Zr/Zr separation distances and Zr-bridging-oxygen bond lengths for the Zr₆(μ₃-O)₄(μ₃-OH)₄ cores of the MOF nodes. Significant differences were evident, however, for apparent separation distances between pairs of nonstructural oxygen atoms. The solved structure for NU-1000-F (FIG. 2B) yielded distances of 2.49 and 2.30 Å—values that are consistent with separation distances within ligated formate, but also with distances between oxygen atoms of terminally ligated and hydrogen-bonded aqua/hydroxo pairs. For NU-1000-FF-Cl (which lacks formate), the solved structure O/O separation distances were 3.24 Å and 3.30 Å (FIG. 2D); these values exceeded the typical upper limit for an aqua/hydroxo pair connected by a hydrogen bond (2.5-3.2 Å). (Jeffrey, G. A., An introduction to hydrogen bonding. Oxford University Press: New York and Oxford, 1997; p 303.) Notably absent from the structure were Zr-ligated chlorides, despite the unambiguous presence of chloride ions in the compound. Rather than formate and H-bonded aqua/hydroxo pairs, the nodes were terminated with aqua/aqua pairs that did not hydrogen-bond to each other and thus displayed greater oxygen/oxygen separation distances. The structure was consistent with hydrogen-bonding by pairs of terminal aqua ligands to water molecules located on their mid-plane with distances of about 3 Å, as shown in FIGS. 2D and 2E. Replacing an aqua/hydroxo ligand pair with an aqua/aqua pair required a charge-compensating anion—ideally, four anions per node, with anions being chlorides in this example. The chloride ions were unable to be located in the crystal structure, implying that they were disordered.

To summarize, the single-crystal X-ray results were consistent with a node structure of Zr₆(μ₃-O)₄(μ₃-OH)₄(H₂O)₈¹²⁺, with eight ligated linker carboxylates partially balancing the 12⁺ charge and four free chlorides completing the charge compensation.

SEM-EDS. For NU-1000-FF-Cl prepared via treatment of the as-synthesized MOF in DMSO+aq. HCl at room temperature, SEM-EDS measurements indicated the presence 3.4 chlorides per node. Treatments with other solvents (+aq. HCl) indicated similar degrees of chloride incorporation.

XPS. XPS measurements likewise indicated the presence of chloride. In reasonable agreement with the SEM-EDS estimate, 3.7 chlorides were found per node, i.e., fairly close to the anticipated (ideal) loading of 4 per node.

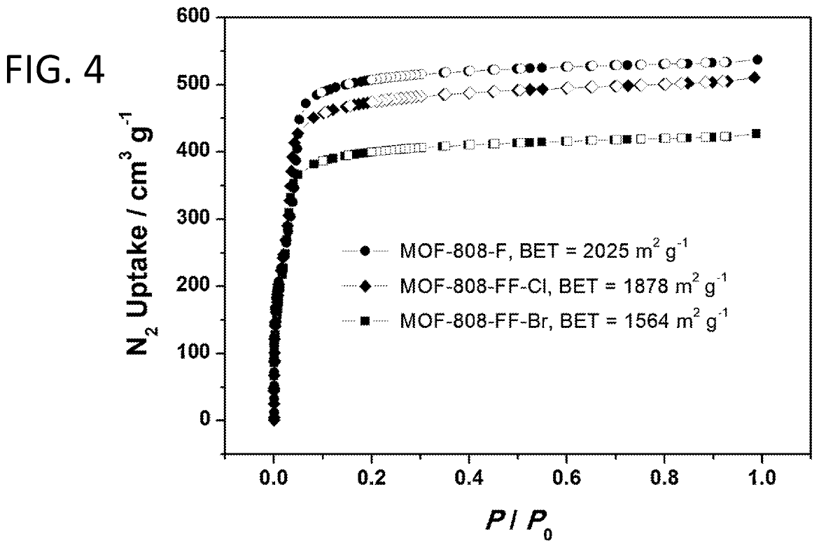

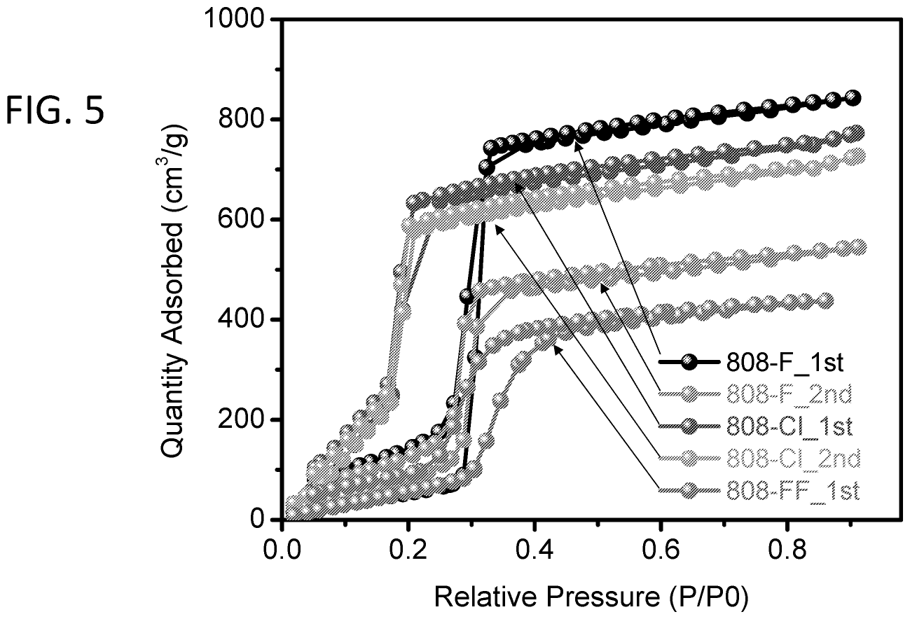

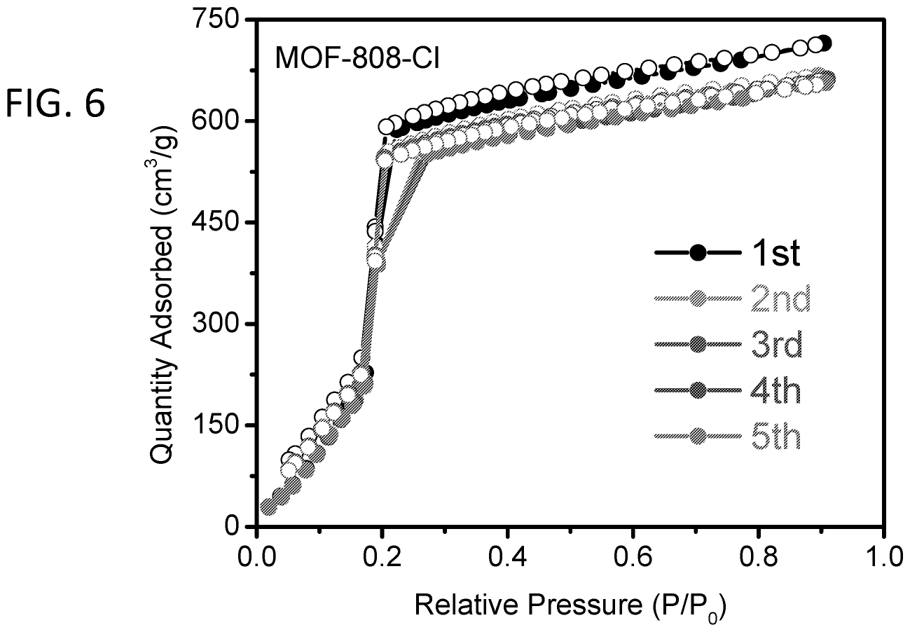

FIG. 4 shows N₂ adsorption isotherms for MOF-808-F, MOF-808-Cl, and MOF-808-Br. FIG. 5 shows water sorption isotherms comparing as-synthesized MOF-808-F to MOF-808-Cl and to a MOF-808 from which both the Cl and formate ligands have been removed (MOF-808-FF). The data show two water-harvesting (uptake and release) cycles. The isotherms show that the MOF-808-Cl MOF exhibits adsorption starting from lower relative humidity, higher maintained absorption capacity, and better recyclability. FIG. 6 shows water sorption isotherms for MOF-808-Cl through 5 cycles. These data demonstrate that MOF-808-Cl can retain its high water uptake capacity for at least five adsorption/desorption cycles.

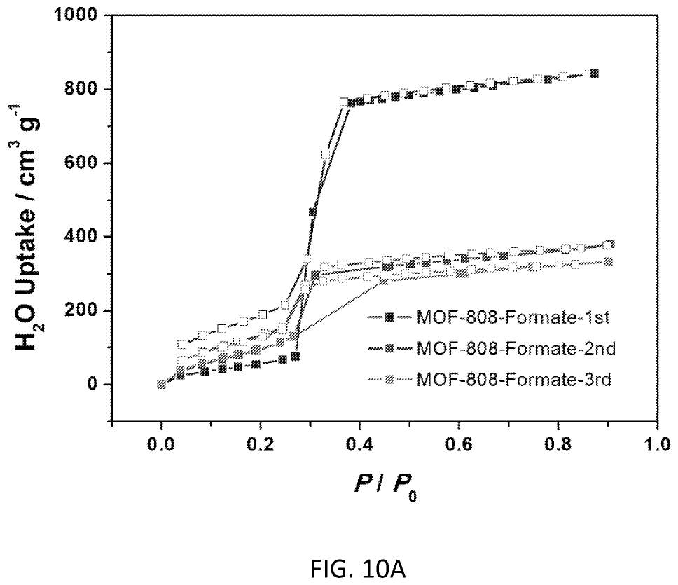

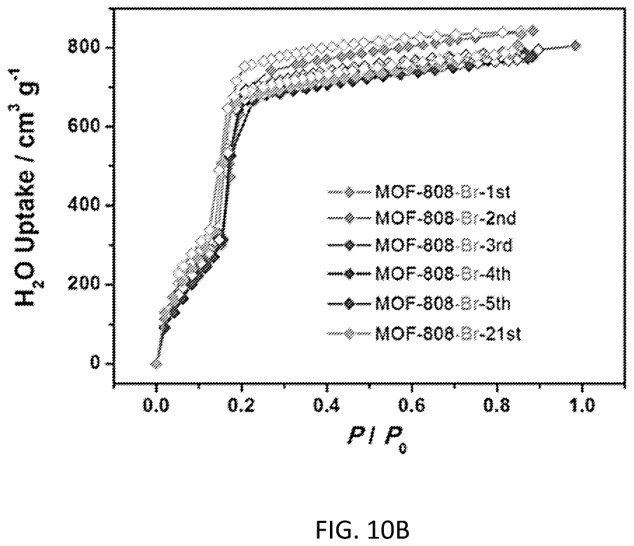

Consistent with unwanted pore-collapse, the high initial water uptake capacity of MOF-808-F, (840 cm³g⁻¹ at the plateau at P/P₀=0.35), faded rapidly—yielding only ˜300 cm³g⁻¹ for cycle 3. In striking contrast was the highly resilient behavior of MOF-808-Br. Plots for cycles 1-5 and 21 evinced that: a) the magnitude of water uptake by MOF-808-Br in the first sorption cycle was similar to that for MOF-808-F at both the onset of the isotherm plateau and near saturation (P/P₀=0.9), despite a greater formula weight; b) uptake and release were sharper, i.e. occurred over a narrower RH (normalized pressure) range; c) the median point for water sorption/desorption was pushed from P/P₀=0.3 for MOF-808-F to P/P₀=0.2 for MOF-808-Br; d) sorption occurred over two distinct regions of pressure, but, essentially zero hysteresis was registered, e) the second and subsequent sorption cycles register almost no capacity decrease relative to the first, illustrating that porosity is maintained and capillary-force-induced pore collapse is absent. FIGS. 10A and 10B show the Isotherms for water uptake and release by: (FIG. 10A) MOF-808-F, 3 cycles; and (FIG. 10B) MOF-808-Br, cycles 1-5 and 21.

To evaluate the potential of MOF-808-Br to generate potable water in desert regions, day/night temperatures and RH values typical of the Arabian and Sonoran deserts were simulated, i.e. 45° C., 5% RH (day) and 25° C., 35% RH (night). From the plots, the working capacity was 600 cm³g⁻¹ (0.48 g·g⁻¹); the corresponding value for MOF-808-Cl was 0.53 g·g⁻¹; 1^{st} cycle.

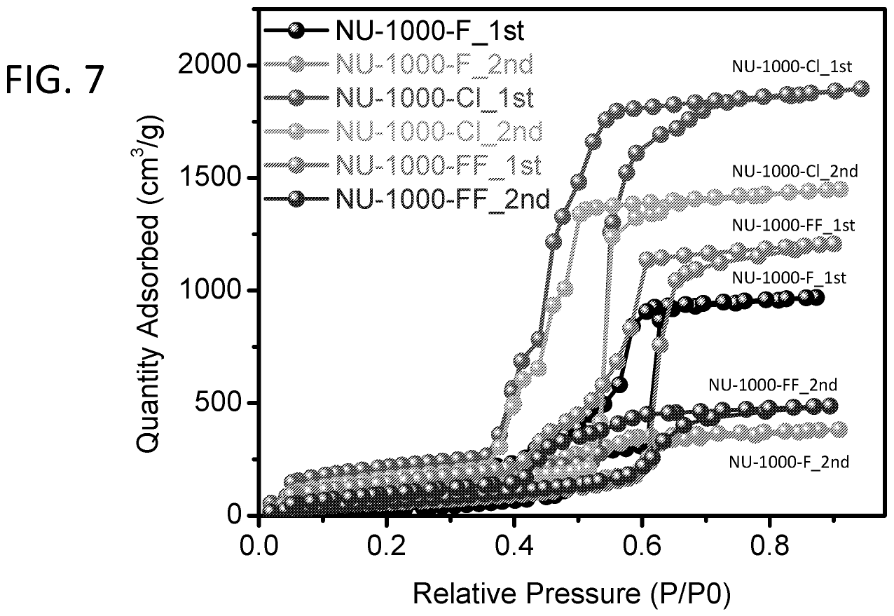

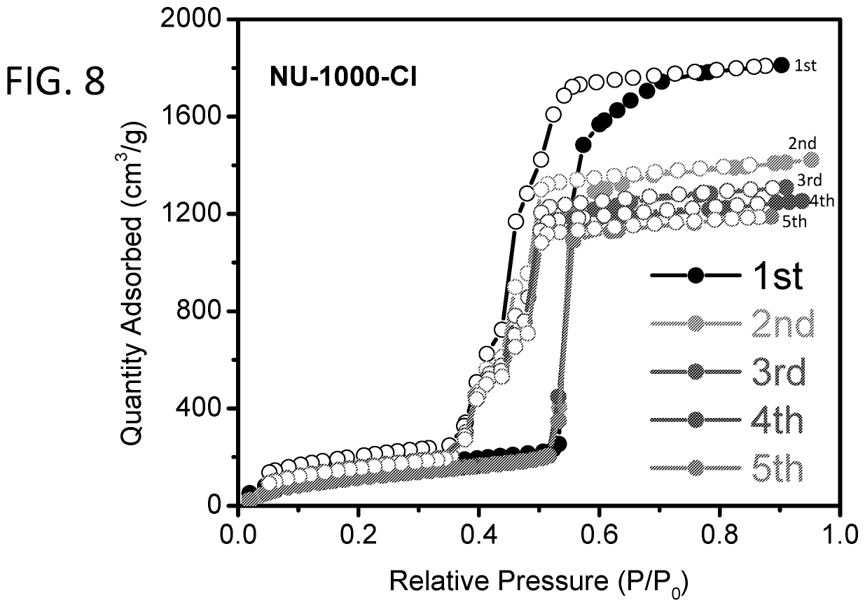

FIG. 7 shows water sorption isotherms comparing as-synthesized NU-1000-F to NU-1000-Cl and to NU-1000 from which both the Cl and formate ligands have been removed (NU-1000-FF). The data show two water-harvesting (uptake and release) cycles. The isotherms show that NU-1000-CL exhibits adsorption starting from lower relative humidity, higher maintained absorption capacity, and better recyclability. FIG. 8 shows water sorption isotherms for NU-1000-Cl through 5 cycles. These data demonstrate that NU-1000-Cl can retain its high water uptake capacity for at least five adsorption/desorption cycles.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” can mean only one or can mean “one or more.” Embodiments of the inventions consistent with either construction are covered.

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A hydrated metal organic framework comprising:
a metal organic framework molecule having positively charged, linker unsaturated nodes and a net positive charge;
water contained within pores of the metal organic framework molecule; and
charge compensating anions that are not bonded to the linker unsaturated nodes via coordination bonds and that have freedom of motion through the pores of the metal organic framework.

2. The hydrated metal organic framework of claim 1, wherein the metal organic framework molecule is a zirconium metal organic framework.

3. The hydrated metal organic framework of claim 1, wherein the net positive electrical charge is imparted by protonation of the linker unsaturated nodes.

4. The hydrated metal organic framework of claim 1, wherein the charge compensating anions are halide anions.

5. The hydrated metal organic framework of claim 4, wherein the halide anions are chloride ions.

6. The hydrated metal organic framework of claim 4, wherein the net positive electrical charge is imparted by protonation of the linker unsaturated nodes.

7. The hydrated metal organic framework of claim 6, wherein the metal organic framework molecule is a zirconium metal organic framework.

8. The hydrated metal organic framework of claim 7, wherein the metal organic framework molecule is NU-1000.

9. The hydrated metal organic framework of claim 7, wherein the metal organic framework molecule is MOF-808.

10. The hydrated metal organic framework of claim 2, wherein the metal organic framework molecule is NU-1000.

11. The hydrated metal organic framework of claim 2, wherein the metal organic framework molecule is MOF-808.

12. A method for harvesting water from an atmosphere containing vapor-phase water molecules using metal organic frameworks, the metal organic frameworks comprising:
metal organic framework molecules having positively charged, linker unsaturated nodes and a net positive charge; and
charge compensating anions that are not bonded to the linker unsaturated nodes via coordination bonds and that have freedom of motion through the pores of the metal organic framework,
the method comprising:
exposing the metal organic frameworks to the atmosphere at a temperature at which vapor-phase water molecules condense as liquid water within the pores of the metal organic framework molecules, wherein the water-soluble ions are solubilized by the liquid water;
increasing the temperature of the metal organic frameworks to a temperature at which the water molecules are released from the pores of the metal organic framework molecules, decreasing the relative humidity of the atmosphere to a relative humidity at which the water molecules are released from the pores of the metal organic framework molecules, or both; and
collecting the released water molecules.

13. The method of claim 12, wherein the metal organic framework molecule is a zirconium metal organic framework.

14. The method of claim 12, wherein the net positive electrical charge is imparted by protonation of the linker unsaturated nodes.

15. The method of claim 12, wherein the charge compensating anions are halide anions.

16. A method of making charge-compensated metal organic frameworks, the method comprising:
synthesizing metal organic framework molecules having linker unsaturated nodes and an initial set of ligands on the linker unsaturated nodes;
dispersing the metal organic framework molecules in a solution comprising a salt that dissociates into cations and charge compensating anions, wherein the cations form coordination bonds to the linker unsaturated nodes to impart a net positive charge to the metal organic framework molecules, and the charge-compensating anions remain solvated; and
separating the positively charged metal organic frameworks and the charge-compensating anions from the solution; and
drying the metal organic framework molecules to provide charge-compensated metal organic frameworks comprising positively charged metal organic framework molecules and charge compensating anions dispersed within pores of the positively charged metal organic framework molecules.

17. The method of claim 16, wherein the initial set of ligands comprises hydroxo ligands, aquo ligands, benzoate ligands, formate ligands, or a combination of two or more thereof.

18. The method of claim 16, wherein the salt is an acid and the cations are protons.

19. The method of claim 18, wherein the acid is hydrochloric acid.

20. The method of claim 16, wherein the metal organic framework is a zirconium metal organic framework.
